(19) United States
(12) Patent Application Publication
(10) Pub. No.: US 2025/0230160 A1
(43) Pub. Date: Jul. 17, 2025

(54) PROCESS FOR THE PREPARATION OF 4-((6BR,10AS)-3-METHYL-2,3,6B,9,10,10A-HEXAHYDRO-1H,7H-PYRIDO[3',4':4,5]PYRROLO[1,2,3-DE]QUINOXALIN-8-YL)-1-(4-FLUORO-PHENYL)-BUTAN-1-ONE AND INTERMEDIATES THEREOF

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Srinivas Reddy GADE, Hyderabad, Telangana (IN); Ravi Kumar BANDI, Hyderabad, Telangana (IN); Venkata Ramana ADI, Hyderabad, Telangana (IN); Srinivas VAKITI, Hyderabad, Telangana (IN); Suresh Kumar Sunkari, Hyderabad, Telangana (IN)

(73) Assignee: MSN LABORATORIES PRIVATE LIMITED, R&D CENTER, Hyderabad, Telangana (IN)

(21) Appl. No.: 18/854,731

(22) PCT Filed: Apr. 10, 2023
(86) PCT No.: PCT/IN23/50347

§ 371 Date: Oct. 7, 2024

(30) Foreign Application Priority Data
Apr. 8, 2022 (IN) 202241021301
Jan. 20, 2023 (IN) 202341003958

(51) Int. Cl. C07D 471/16

(52) U.S. Cl. CPC C07D 471/16

(57) ABSTRACT

The present invention relates to a process for the preparation of 4-((6bR,10aS)-3-methyl-2,3,6b,9,10,10a-hexahydro-1H,7H-pyrido[3′,4′:4,5]pyrrolo[1,2,3-de]quinoxalin-8-yl)-1-(4-fluoro-phenyl)-butan-1-one represented by the following structural formula-1 and pharmaceutically acceptable salts thereof.

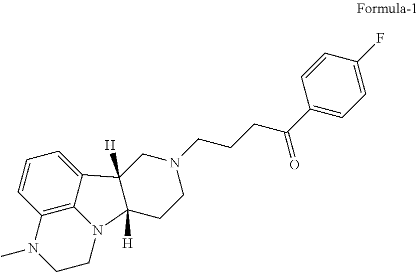 [SMILES: CN1CCN2c3c(cccc31)[C@@H]1CN(CCCC(=O)c3ccc(F)cc3)CC[C@@H]12]

The present invention further relates to processes for the preparation of intermediates of 4-((6bR,10aS)-3-methyl-2,3,6b,9,10,10a-hexahydro-1H,7H-pyrido[3′,4′:4,5]pyrrolo[1,2,3-de]quinoxalin-8-yl)-1-(4-fluoro-phenyl)-butan-1-one compound of formula-1.

The present invention further relates to novel intermediates of 4-((6bR,10aS)-3-methyl-2,3,6b,9,10,10a-hexahydro-1H,7H-pyrido[3′,4′:4,5]pyrrolo[1,2,3-de]quinoxalin-8-yl)-1-(4-fluoro-phenyl)-butan-1-one compound of formula-1 and process for preparation thereof.

### RELATED APPLICATIONS

This application claims the benefit of priority of our Indian patent applications 202241021301 filed on Apr. 8, 2022 and 202341003958 filed on Jan. 20, 2023 which are incorporated herein by reference.

### FIELD OF THE INVENTION

The present invention provides a process for the preparation of 4-((6bR,10aS)-3-methyl-2,3,6b,9,10,10a-hexahydro-1H,7H-pyrido[3′,4′:4,5]pyrrolo[1,2,3-de]quinoxalin-8-yl)-1-(4-fluoro-phenyl)-butan-1-one represented by the following structural formula-1, its salts and intermediates thereof.

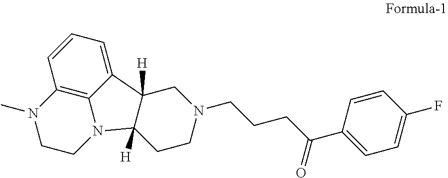 [SMILES: CN1CCN2c3c(cccc31)[C@@H]1CN(CCCC(=O)c3ccc(F)cc3)CC[C@@H]12]

### BACKGROUND OF THE INVENTION

4-((6bR,10aS)-3-Methyl-2,3,6b,9,10,10a-hexahydro-1H,7H-pyrido[3′,4′:4,5]pyrrolo [1,2,3-de]quinoxalin-8-yl)-1-(4-fluoro-phenyl)-butan-1-one 4-methylbenzene sulfonate is commonly known as Lumateperone tosylate.

Lumateperone tosylate is designed and developed by Intra-Cellular Therapies Inc., which is approved by USFDA on Dec. 20, 2019. It is an atypical antipsychotic indicated for the treatment of schizophrenia in adults and is being marketed under the brand name CAPLYTA.

USRE39680E describes Lumateperone, its intermediates and processes for preparation thereof.

U.S. Pat. No. 8,648,077 B2 describes two crystalline polymorphic forms viz., Form-A and Form-B of Lumateperone tosylate and processes for preparation thereof.

Still, there is a need to develop an improved process for the preparation of Lumateperone, its salts (for example tosylate) and intermediates thereof.

### BRIEF DESCRIPTION OF THE INVENTION

The first embodiment of the present invention is to provide a process for the preparation of (6bR,10aS)-3-methyl-6b,7,8,9,10,10a-hexahydro-1H-pyrido[3′,4′:4,5]pyrrolo [1,2,3-de]quinoxalin-2(3H)-one compound of formula-14.

The second embodiment of the present invention is to provide a process for the preparation of compound of formula-1 and pharmaceutically acceptable salts thereof.

The third embodiment of the present invention is to provide a process for the preparation of compound of formula-15 and salts thereof.

The fourth embodiment of the present invention is to provide a process for the preparation of compound of formula-1 and pharmaceutically acceptable salts thereof.

### DETAILED DESCRIPTION OF THE INVENTION

The “solvent” used in the present invention can be selected from but not limited to “hydrocarbon solvents” such as n-pentane, n-hexane, n-heptane, cyclohexane, petroleum ether, benzene, toluene, xylene and the like; “ether solvents” such as dimethyl ether, diethyl ether, diisopropyl ether, methyl tert-butyl ether, 1,2-dimethoxyethane, tetrahydrofuran, 1,4-dioxane and the like; “ester solvents” such as methyl acetate, ethyl acetate, n-propyl acetate, isopropyl acetate, n-butyl acetate, isobutyl acetate, tert-butyl acetate and the like; “polar-aprotic solvents” such as dimethylacetamide, dimethylformamide, dimethylsulfoxide, N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcohol solvents” such as methanol, ethanol, n-propanol, iso-propanol, n-butanol, iso-butanol, 2-butanol, tert-butanol, ethane-1,2-diol, propane-1,2-diol and the like; “polar solvents” such as water; formic acid, acetic acid and the like or mixture of any of the afore mentioned solvents.

The “base” used in the present invention can be selected from but not limited to “inorganic bases” selected from “alkali metal carbonates” such as sodium carbonate, potassium carbonate, lithium carbonate, cesium carbonate and the like; “alkali metal bicarbonates” such as sodium bicarbonate, potassium bicarbonate, lithium bicarbonate, cesium bicarbonate and the like; “alkali metal hydroxides” such as sodium hydroxide, potassium hydroxide, lithium hydroxide, cesium hydroxide and the like; “alkali metal hydrides” such as sodium hydride, potassium hydride, lithium hydride and the like; “alkali metal amides” such as sodium amide, potassium amide, lithium amide and the like; ammonia; “organic bases” like “alkali metal alkoxides” such as sodium methoxide, sodium ethoxide, potassium methoxide, potassium ethoxide, lithium methoxide, lithium ethoxide, sodium tert.butoxide, potassium tert.butoxide, lithium tert.butoxide and the like; alkali metal and alkali earth metal salts of acetic acid such as sodium acetate, potassium acetate, magnesium acetate, calcium acetate and the like; dimethylamine, diethylamine, diisopropyl mine, diisopropylethylamine (DIPEA), diisobutylamine, trimethylamine, triethylamine, triisopropylamine, tributylamine, tert.butyl amine, pyridine, piperidine, 4-dimethylamino pyridine (DMAP), quinoline, imidazole, N-methylimidazole, 1,8-diazabicyclo[5.4.0]undec-7-ene (DBU), 1,5-diazabicyclo[4.3.0]non-5-ene (DBN), dimethylaniline, N-methylmorpholine (NMM), 1,4-diazabicyclo[2.2.2]octane (DABCO), 2,6-lutidine and the like; “organolithium bases” such as methyl lithium, n-butyl lithium, lithium diisopropylamide (LDA) and the like; “organosilicon bases” such as lithium hexamethyldisilazide (LiHMDS), sodium hexamethyldisilazide (NaHMDS), potassium hexamethyldisilazide (KHMDS) and the like or mixtures thereof.

The “acid” used in the present invention refers to inorganic acid selected from hydrochloric acid, hydrobromic acid, sulfuric acid, nitric acid, phosphoric acid, sulfamic acid and the like; organic acid such as formic acid, acetic acid, trifluoroacetic acid, methanesulfonic acid, ethanesulfonic acid, benzenesulfonic acid, trifluoromethane sulfonic acid, p-toluenesulfonic acid, tartaric acid, mandelic acid, malic acid, oxalic acid, formic acid, ascorbic acid, phosphorous acid, maleic acid, succinic acid, malonic acid, oxalic acid, dibenzoyl tartaric acid, lactic acid, cinnamic acid and the like

The “nitrosation reagent” used in the present invention can be selected from but not limited to sodium nitrite, potassium nitrite, calcium nitrite, cupric nitrite, tert-butyl nitrite (TBN), isonitropropane, nitromethane, nitrous acid, nitrating mixture (a mixture of nitric acid and sulfuric acid or a mixture of nitric acid and acetic acid and the like or mixtures thereof.

The “methylating agent or N-methylating agent” used in the present invention can be selected from methyl halides such as methyl iodide, dimethyl sulfate (DMS), dimethyl carbonate, trimethyloxonium tetrafluoroborate (Me₃O·BF₄), trimethylphosphate, methyl alkyl/aryl sulfonates such as methyl methane sulfonate (MeOMs), methyl ethanesulfonate, methyl benzenesulfonate, methyl p-toluene sulfonate (MeOTs), methyl trifluoromethanesulfonate (MeOTf), trimethylsilyl diazomethane (TMSD), tetramethylammonium salts such as tetramethylammonium halides and the like.

In the present invention, PG′ represents “N-protecting group” or “amine protecting group” selected from but not limited to alkoxycarbonyl such methoxycarbonyl (Moc), ethoxycarbonyl, tert-butyloxycarbonyl (Boc), benzyloxycarbonyl (Cbz), p-methoxybenzyl carbonyl (Moz or MeOZ), 9-fluorenylmethyloxy carbonyl (Fmoc), acetyl (Ac), benzoyl (Bz), benzyl (Bn), carbamate group, p-methoxyphenyl (PMP), p-methoxybenzyl (PMB), 3,4-dimethoxybenzyl (DMPM), tosyl (Ts), trifluoroacetyl (TFA), trichloroethoxycarbonyl (Troc), pivaloyl (Piv), triphenylmethyl (trityl or Trt) and the like.

The “alkali metal halide” used in the present invention can be selected from but not limited to NaCl, NaBr, NaI, KCl, KBr, KI, CsCl, CsBr, CsI and the like.

In the present invention, the word “resolution” means a process of separation of a racemic mixture into its respective enantiomers.

The first embodiment of the present invention provides a process for the preparation of compound of formula-14, comprising resolution of compound of formula-11.

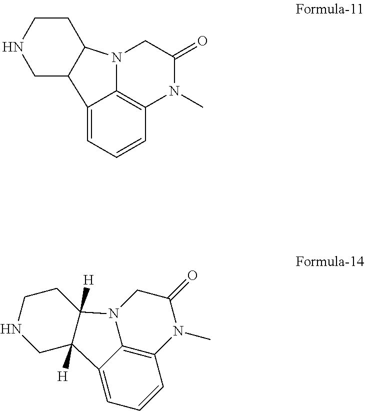 [SMILES: CN1C(=O)CN2c3c(cccc31)C1CNCCC12 ; CN1C(=O)CN2c3c(cccc31)[C@@H]1CNCC[C@@H]12]

In the above process, the resolution step is carried out by using a chiral acid in a solvent to provide corresponding diastereomeric salt and then treating the said salt with a base in a solvent to provide compound of formula-14.

The “chiral acid” in the present invention wherever used can be selected from but not limited to (2S,3S)-2,3-bis(benzoyloxy)-4-(dimethylamino)-4-oxobutanoic acid, (2S,3S)-2,3-bis(benzoyloxy)-4-(diethylamino)-4-oxobutanoic acid, (2S,3S)-2,3-bis(benzoyloxy)-4-oxo-4-(pyrrolidin-1-yl)butanoic acid, (2S,3S)-4-(isopropylamino)-2,3-bis((4-methyl benzoyl)oxy)-4-oxobutanoic acid, (2S,3S)-2,3-bis(benzoyloxy)-4-(isopropylamino)-4-oxobutanoic acid, mandelic acid, acetyl mandelic acid, tartaric acid, di-p-tolyl tartaric acid, dibenzoyl tartaric acid, camphor sulfonic acid and the like.

The base is selected from inorganic bases and organic bases.

The solvent is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

The process according to the first embodiment of the present invention further comprising;
a) reducing compound of formula-14 with a reducing agent in a solvent to provide compound of formula-15, which is optionally converted to its acid-addition salt by treating with an acid in a solvent,

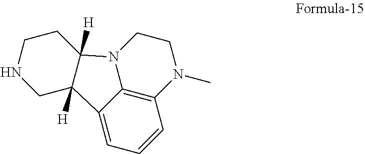 [SMILES: CN1CCN2c3c(cccc31)[C@@H]1CNCC[C@@H]12]

b) reacting compound of formula-15 or its salt with compound of formula-16 to provide compound of formula-1,

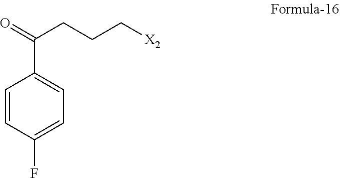

wherein, ‘X₂’ selected from halogen such as F, Cl, Br, I;
c) treating compound of formula-1 with an acid in a solvent to provide corresponding acid-addition salt.

The reducing agent in step-a) can be selected from Borane-Dimethyl sulfide (Borane-DMS), Borane-Tetrahydrofuran (Borane-THF), Lithium aluminium hydride (LiAlH₄), NaBH₄, sodium cyanoborohydride, Aluminium hydride (AlH₃), lithium trialkoxyaluminium hydrides such as lithium triethoxyaluminium hydride (Li(EtO)3AlH), lithium tri tert.butoxyaluminium hydride (Li(OtBu)3AlH), diisobutylaluminium hydride (DIBAL), tetramethylammonium triacetoxyborohydride, triethylsilane, NaAlH(O-t-Bu)₃, Na(AcO)₃BH, B₂H₆, sodium bis(2-methoxyethoxy)aluminumhydride (Red-Al or Vitride), catalytic hydrogenation in presence of Pd, Pt, Rh, Zn, Raney Ni, PtO₂ and the like; Fe, Fe in acidic media like NH₄Cl, HCl, acetic acid; Sn in acidic media like HCl; Zn, Zn in acidic media like HCl, NH₄Cl, acetic acid, formic acid or ammonium formate.

The acid is selected from inorganic acids selected from those defined as above.

In step-b) the reaction is carried out in presence of a base selected from organic bases and inorganic bases or mixtures thereof optionally in presence of an alkali metal halide selected from those as described above;

In step-c) the acid is selected from those as defined above; preferably p-toluenesulfonic acid;

In step-a) to step-c) the solvent wherever necessary can be selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

The first aspect of the first embodiment of the present invention provides a process for the preparation of compound of formula-14, comprising resolution of compound of formula-11 with a chiral acid compound of formula-12 in presence of a solvent.

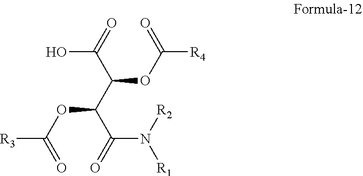

wherein, ‘R₁’ and ‘R₂’ are independently H, lower alkyl, or lower cycloalkyl; or ‘R₁’ and ‘R₂’ together with the Nitrogen atom to which they are attached form an optionally substituted 3-6 membered saturated heterocyclic ring optionally containing 1 or 2 additional heteroatoms selected from S or O; ‘R₃’ and ‘R₄’ are independently phenyl or 5-6 membered heteroaryl and is optionally substituted by halogen, hydroxyl, nitro, lower alkyl, or lower cycloalkyl.

In the present invention, the above resolution process comprising the steps of reacting compound of formula-11 with chiral acid compound of formula-12 in presence of a solvent to provide compound of formula-13, and treating compound of formula-13 with a base to provide compound of formula-14.

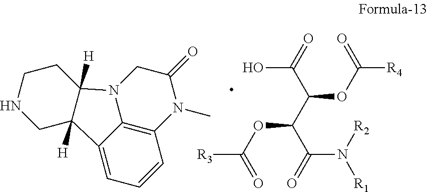

wherein, R₁′, ‘R₂’, ‘R₃’ and ‘R₄’ are as defined above; In the above described process, the chiral acid, the solvent and the base are selected from those as defined above.

The first aspect of the first embodiment of the present invention specifically provides a process for the preparation of compound of formula-14, comprising reaction of compound of formula-11 with (2S,3S)-2,3-bis(benzoyloxy)-4-(dimethylamino)-4-oxobutanoic acid compound of formula-12a in presence of a solvent to provide compound of formula-13a

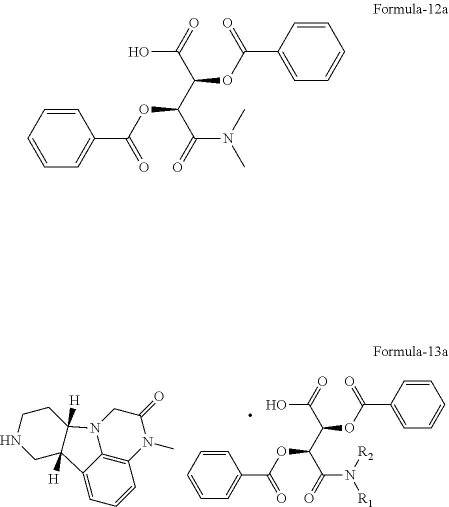

and then treating compound of formula-13a with a base in presence of a solvent to provide compound of formula-14.

The base and the solvent in the above process are selected from those defined above.

In an aspect of the present invention, the chiral acid compound of formula-12 can be selected from but not limited to the following compounds;

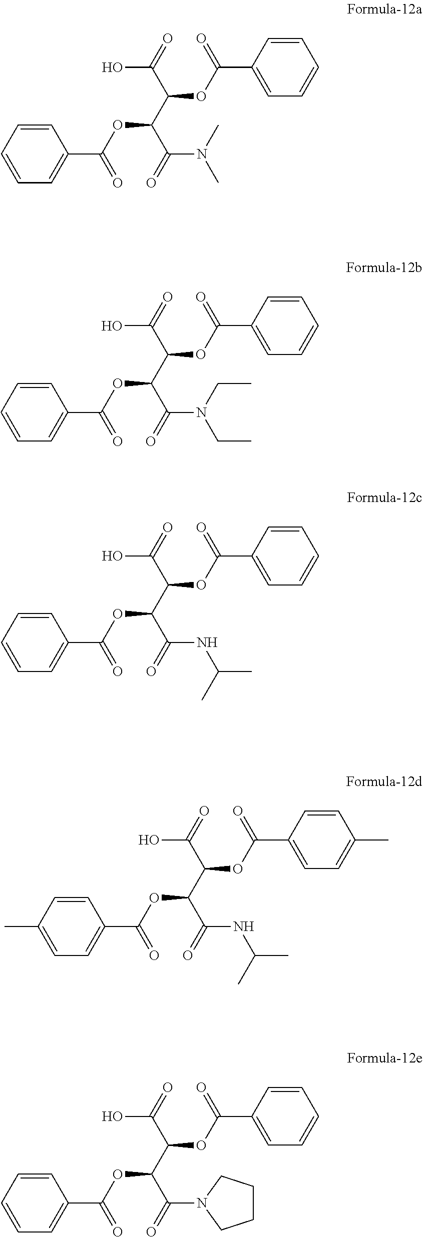

The second embodiment of the present invention provides a process for the preparation of compound of formula-1 and its pharmaceutically acceptable salts, comprising;
a) resolution of compound of formula-11 with a chiral acid compound of formula-12 to provide compound of formula-14,
b) reduction of compound of formula-14 with a reducing agent in a solvent to provide compound of formula-15, which is optionally converted to its acid-addition salt by treating with an acid in a solvent,
c) reacting compound of formula-15 with compound of formula-16 to provide compound of formula-1,
d) treating compound of formula-1 with an acid to provide its pharmaceutically acceptable acid-addition salt.

The resolution process described in step-a) is carried out according to the process described in first embodiment of the present invention;

The conversion of compound of formula-14 to compound of formula-1 and its salts is carried out according to the process described under first embodiment of the present invention.

The compound of formula-11 which is used as starting material in the present invention can be prepared by any of the processes known in the art.

The compound of formula-11 which is used in the present invention can also be prepared by the process as described in the present invention which comprising the steps of;
a) reacting benzene-1,2-diamine compound of formula-2 with compound of formula-3 in presence of a base to provide 3,4-dihydroquinoxalin-2(1H)-one compound of formula-4,

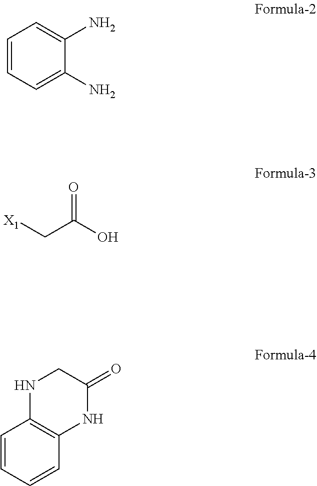

wherein, ‘X₁’ selected from halogen such as F, Cl, Br, I;
b) treating compound of formula-4 with a nitrosation agent to provide 4-nitroso-3,4-dihydroquinoxalin-2(1H)-one compound of formula-5,

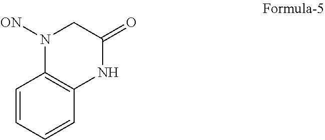

c) reduction of compound of formula-5 with a reducing agent to provide 4-amino-3,4-dihydroquinoxalin-2(1H)-one compound of formula-6,

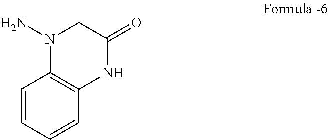 [SMILES: NN1CC(=O)Nc2ccccc21]

d) reacting compound of formula-6 with compound of formula-7 to provide compound of formula-8,

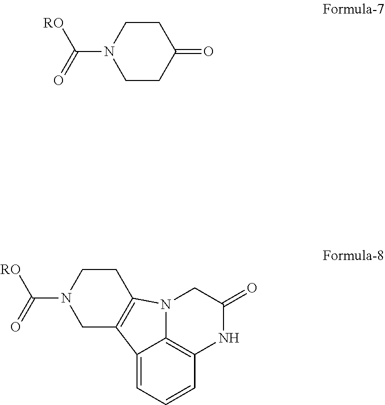

wherein, ‘R’ is selected from C₁-C₆ straight chain or branched chain alkyl, aryl or aralkyl;
e) treating compound of formula-8 with a reducing agent to provide compound of formula-9,

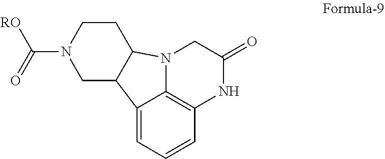

f) reacting compound of formula-9 with a methylating agent to provide compound of formula-10,

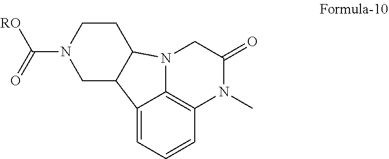

g) treating compound of formula-10 with an inorganic base to provide compound of formula-11.

In the above process, step-a) is carried out in presence of a base selected from organic bases and inorganic bases or mixture thereof;

The nitrosation agent in step-b) is selected from those as defined above in the present invention;

The reducing agent in step-c) is selected from but not limited to Fe, Fe in acidic media like NH₄Cl, HCl, acetic acid; Sn in acidic media like HCl; Zn, Zn in acidic media like HCl, NH₄Cl, acetic acid, formic acid or ammonium formate; Pd, Pt, Rh, Raney Ni, NaBH₄ optionally in combination with catalytic amount of NiCl2.6H2O or COCl2.6H2O, Lithium borohydride, diborane, sodium aluminium hydride, lithium aluminum hydride, hydrazine hydrate, sodium dithionate, sodium sulfide, sodium amalgam, platinum oxide, trialkylsilane, titanium trichloride, ammonium sulfide, borane-DMS, borane-tetrahydrofuran and the like optionally in combination with hydrogen.

The reaction in step-d) is carried out in presence of an acid selected from inorganic acids described above;

The reducing agent in step-e) is selected from but not limited to diisobutylaluminum hydride (DIBAL-H), sodium triacetoxyborohydride, lithium trialkoxyaluminium hydrides such as lithium triethoxyaluminium hydride (Li(EtO)3AlH), lithium tri tert.butoxy aluminium hydride (Li(OtBu)3AlH), sodium borohydride, sodium cyanoborohydride, triethylsilane, lithium aluminum hydride, sodium bis(2-methoxyethoxy)aluminium hydride (Vitride), borane-tetrahydrofuran, borane-dimethylsulfide and the like;

The methylating agent in step-f) can be selected from those as defined above in the present invention;

The inorganic base in step-g) is selected from those defined above and the reaction mixture is heated to a temperature ranging from 30° C. to 150° C.;

The solvent in step-a) to step-g) wherever necessary can be selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

The present invention provides a novel intermediate compound represented by the following structural formula-14. This intermediate is useful for the preparation of compound of formula-1 and its pharmaceutically acceptable salts such as tosylate salt.

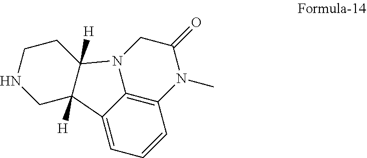 [SMILES: CN1C(=O)CN2c3c(cccc31)[C@@H]1CNCC[C@@H]12]

An aspect of the present invention provides the use of intermediate compound of formula-14 for the preparation of Lumateperone and its pharmaceutically acceptable salts.

The present invention further provides a process for the preparation of compound of formula-15, comprising reducing compound of formula-14 with a reducing agent in a solvent.

The reducing agent and the solvent are same as defined in step-a) of the first embodiment of the present invention.

The third embodiment of the present invention provides a process for the preparation of compound of formula-15 and salts thereof, comprising,
a) treating compound of formula-11 with D(−)-tartaric acid in presence of a solvent to provide corresponding tartrate salt having the following structural formula

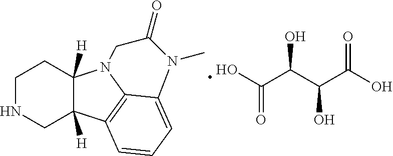

b) treating the compound obtained in step-a) with a base in a solvent to provide compound of formula-14,
c) reducing compound of formula-14 with a reducing agent in a solvent to provide compound of formula-15, which is optionally converted into its salt by treating with an acid in a solvent.

The base in step-b) is selected from inorganic bases, organic bases defined above.

The reducing agent in step-c) is same as defined in step-a) of the first aspect of the first embodiment of the present invention.

The acid in step-c) is selected from but not limited to inorganic acids selected from those defined as above for example HCl, HBr and the like.

The solvent used in step-a) to step-c) of the above process is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

The fourth embodiment of the present invention provides a process for the preparation of compound of formula-1, comprising;
a) treating compound of formula-11 with D(−)-tartaric acid in a solvent to provide corresponding tartrate salt,
b) treating the tartrate salt with a base in a solvent to provide compound of formula-14,
c) reducing compound of formula-14 with reducing agent in a solvent to provide compound of formula-15, which is optionally converted to its salt by treating with an acid in a solvent,
d) reacting compound of formula-15 or its salt with compound of formula-16 to provide compound of formula-1,
e) treating compound of formula-1 with an acid to provide corresponding acid-addition salt.

Various reactions/conversions mentioned in fourth embodiment of the present invention utilizes the similar reagents and solvents defined for similar kind of conversions in the afore mentioned embodiments.

The present invention further provides racemization of (6bS,10aR)-3-methyl-6b,7,8,9,10,10a-hexahydro-1H-pyrido[3′,4′:4,5]pyrrolo[1,2,3-de]quinoxalin-2(3H)-one compound of formula-17 (which is an unwanted isomer) to provide compound of formula-11, comprising;
a) reacting compound of formula-17 with an amine protecting agent in a solvent to provide compound of formula-18,

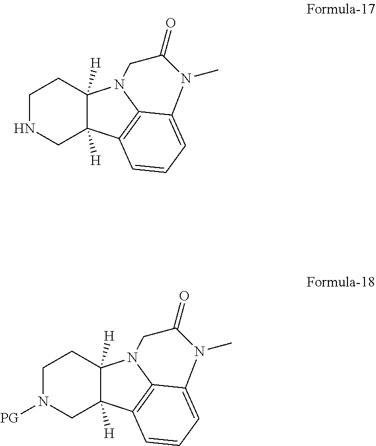

b) treating compound of formula-18 with an oxidizing agent in a solvent to provide compound of formula-19,

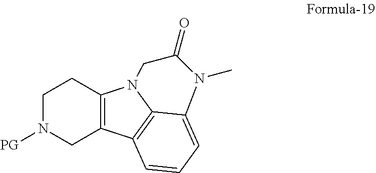

c) treating compound of formula-19 with a reducing agent to provide compound of formula-20,

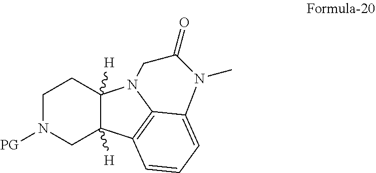

d) deprotecting compound of formula-20 with a deprotecting agent to provide compound of formula-11.

The amine protecting agent in step-a) is selected from trityl halides such as trityl chloride, 4-methyl trityl chloride; alkoxy carbonyl halide and anhydrides such as methoxy carbonyl halide, ethoxy carbonyl halide, tert.butoxy carbonyl halides, di-tert.butyl dicarbonate (DIBOC); aryloxy carbonyl halides; arylalkoxy carbonyl halides such as benzyl chloroformate, ethyl chloroformate, fluorenylmethyloxy carbonyl chloride (FMOC chloride); straight chain or branched chain alkyl halides such as methyl halides; straight chain or branched chain alkenyl halides such as allyl halides, substituted or unsubstituted acids, acid halides and acid esters such as acetyl halide, chloro acetyl halide, pivaloyl halides, trichloroacetyl halides, trifluoro acetyl halides, alkyl trifluoroacetates; substituted or unsubstituted acid anhydrides such as acetic anhydride, chloro acetic anhydride, trifluoro acetic anhydride, aryl alkyl halides, alkoxyalkyl halide, aryloxy alkyl halide, (alkyl)m(aryl)₃₋ₘ silyl halides and triflates; alkyl or aryl sulfonyl halides or anhydrides such as mesyl halides, mesyl anhydride, tosyl halides, tosyl anhydrides, benzyl halides, benzoyl halides and the like.

The “oxidizing agent” in step-b) is selected from sodium hypochlorite in presence of a catalyst like TEMPO (2,2,6,6-tetramethyl-1-piperidinyloxy, free radical)/KBr; hydrogen peroxide, NBS-benzoyl peroxide, cumene hydroperoxide, per acids such as peracetic acid, trifluoro peracetic acid, perbenzoic acid, m-chloroperbenzoic acid (MCPBA); 4,5-dichloro-3,6-dihydroxyphthalonitrile (DDQ), 2,3,5,6-tetrachlorocyclohexa-2,5-diene-1,4-dione (Chloranil), potassium permanganate (KMnO₄), manganese dioxide and the like.

The reducing agent in step-c) is selected from but not limited to diisobutylaluminum hydride (DIBAL-H), sodium triacetoxyborohydride, lithium trialkoxyaluminium hydrides such as lithium triethoxyaluminium hydride (Li(EtO)3AlH), lithium tri tert.butoxy aluminium hydride (Li(OtBu)3AlH), sodium borohydride, sodium cyanoborohydride, triethylsilane, lithium aluminum hydride, sodium bis(2-methoxyethoxy)aluminium hydride (Vitride), borane-tetrahydrofuran, borane-dimethylsulfide and the like;

The “deprotecting agent” in step-d) is selected based on the protecting group employed. The “deprotecting agent” is selected from but not limited to acids such as hydrochloric acid, hydrobromic acid, sulfuric acid, nitric acid, aq.phosphoric acid, trifluoroacetic acid, methanesulfonic acid, p-toluenesulfonic acid; acetyl chloride in combination with alcohols; bases such as alkali metal hydroxides, alkali metal carbonates, cesium carbonate/imidazole, alkali metal bicarbonates, ammonia, aqueous ammonia, ammonium cerium(IV) nitrate (CAN); and organic bases such as methylamine, ethylamine, diethylamine, triethylamine, piperidine; hydrogenating agents such as Pd/C, Pd(OH)₂/C (Pearlman's catalyst), palladium acetate, platinum oxide, platinum black, Rh/C, Ru, sodium borohydride, Na-liquid ammonia, Raney-Ni, Zn-acetic acid, tri(C₁-C₆)alkylsilanes, tri(C₁-C₆) alkylsilyl halides and the like.

In one of the aspects of the above described process, the deprotecting agent is a base selected from the bases defined in the present invention.

The solvent used in step-a) to step-d) of the above process is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

The compound of formula-2, compound of formula-3a, compound of formula-7a and compound of formula-16a which are used in the present invention can be prepared by any of the processes known in the art or they can be procured from any of the sources available.

The compound of formula-1a produced by the process of the present invention is having particle size distribution of D₉₀ less than about 400 μm, preferably less than about 300 μm, more preferably less than about 200 μm.

In one aspect of the present invention, the compound of formula-1a is having particle size distribution of D₉₀ less than about 100 μm, preferably less than about 50 μm.

The compound of formula-1a produced by the process of the present invention can be further micronized or milled to get desired particle size to achieve desired solubility profile based on different forms of pharmaceutical composition requirements. Techniques that may be used for particle size reduction includes but not limited to single or multi-stage micronization using cutting mills, pin/cage mills, hammer mills, jet mills, fluidized bed jet mills, ball mills and roller mills. Milling/micronization may be performed before drying or after drying of the product.

The present invention is schematically represented as follows:

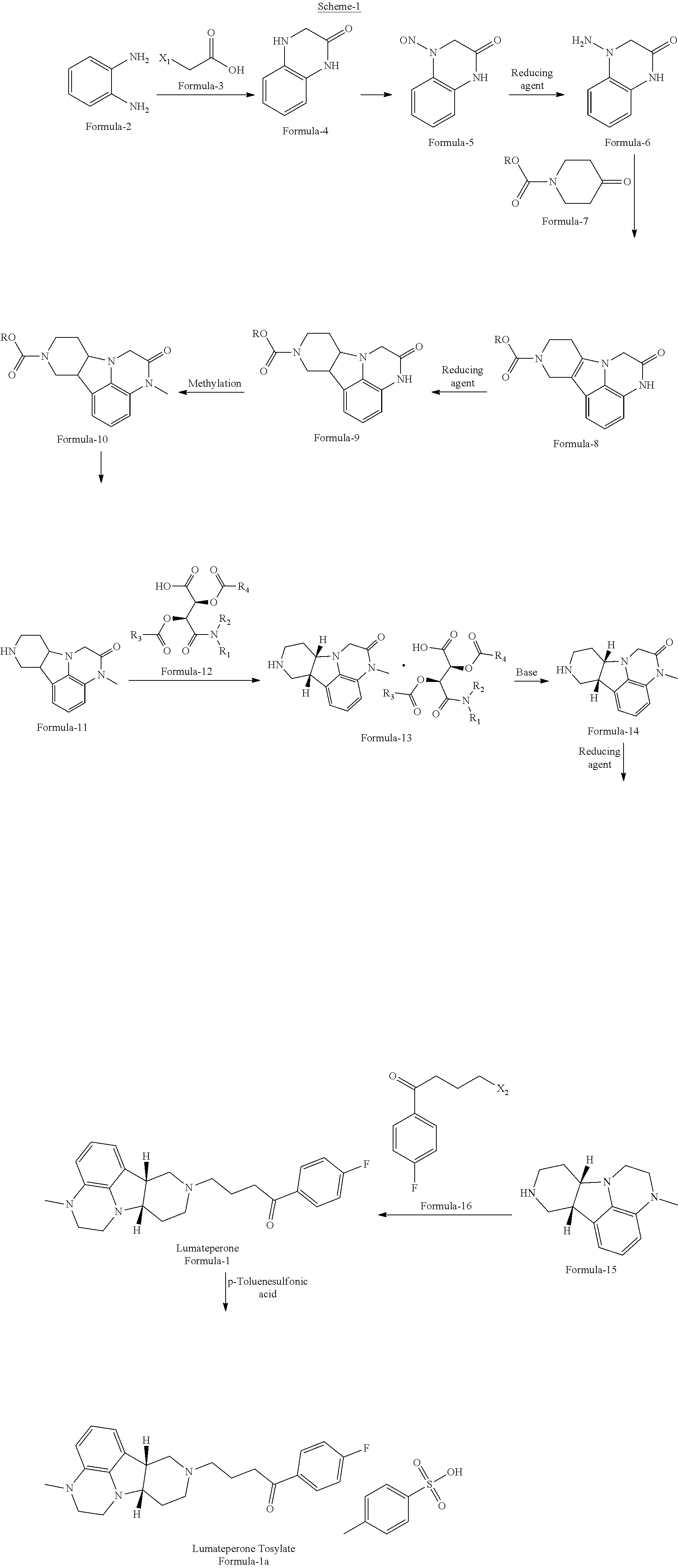

Wherein, ‘X₁’ and ‘X₂’ are each independently selected from halogens such as F, Cl, Br, I; ‘R’ is selected from C₁-C₆ straight chain or branched chain alkyl, aryl or aralkyl groups; ‘R₁’ and ‘R₂’ are independently H, lower alkyl, or lower cycloalkyl; or ‘R₁’ and ‘R₂’ together with the ‘N’ to which they are attached form an optionally substituted 3 to 6 membered saturated heterocyclic ring optionally containing 1 or 2 additional heteroatoms selected from S or O; ‘R₃’ and ‘R₄’ are independently phenyl or 5-6 membered heteroaryl that is optionally substituted by halogen, hydroxyl, nitro, lower alkyl, or lower cycloalkyl.

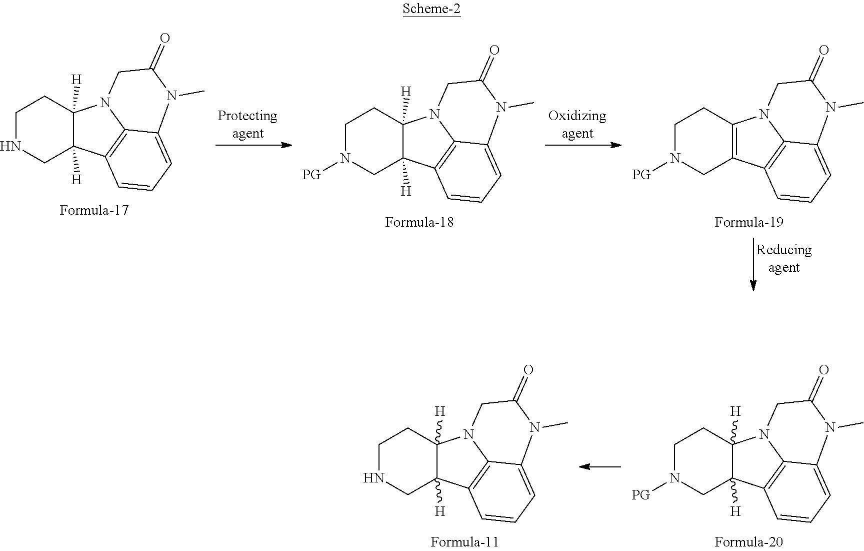

In the above scheme, “PG” represents amine protecting group.

The best mode of carrying out the present invention is illustrated by the below mentioned examples. These examples are provided as illustration only and hence should not be construed as limitation to the scope of the invention.

### EXAMPLES

### Example-1: Preparation of 3,4-dihydroquinoxalin-2(1H)-One (Formula-4)

2-Chloroacetic acid compound of formula-3a (524.5 gm) was added lot wise to a mixture of sodium bicarbonate (582.7 gm) and water (2500) at 15-20° C. Benzene-1,2-diamine compound of formula-2 (500 gm) was added to the reaction mixture at 15-20° C. Heated the reaction mixture to 90-95° C. and stirred for 6 hr 50 min at the same temperature. Cooled the reaction mixture to 25-30° C. and stirred for 12 hr 30 min at the same temperature. Further cooled the reaction mixture to 0-5° C. and stirred for 2 hr 15 min at same temperature. Filtered the solid, washed with water and dried to get the title compound. Yield: 424 gm.

### Example-2: Preparation of 4-nitroso-3,4-dihydroquinoxalin-2(1H)-One (Formula-5)

Water (400 ml) was added to a mixture of compound of formula-4 (400 gm) and acetic acid (2400 ml) at 25-30° C. and stirred the reaction mixture for 35 min at the same temperature. Cooled the mixture to 0-5° C. and stirred for 10 min at the same temperature. Aq. sodium nitrite solution (242.1 gm of sodium nitrite in 400 ml of water) was slowly added to the reaction mixture at 0-5° C. and stirred for 3 hr 45 min at the same temperature. Filtered the solid, washed with water and dried to get the title compound. Yield: 418 gm.

### Example-3: Preparation of ethyl 2-oxo-2,3,9,10-tetrahydro-1H-pyrido[3′,4′:4,5]pyrrolo

[1,2,3-de]quinoxaline-8(7H)-carboxylate (Formula-8a) Step-a: Ammonium chloride (241 gm) was added to a mixture of methanol (4000 ml) and compound of formula-5 (200 gm) at 25-30° C. Cooled the reaction mixture to 15-25° C. Zinc dust (148 gm) was slowly added lot wise to the reaction mixture at 15-25° C. for 1 hr and stirred the reaction mixture for 45 min at the same temperature. Ammonium chloride (60.2 gm) was added to the reaction mixture at 25-30° C. and stirred for 2 hr 20 min at the same temperature. Heated the reaction mixture to 45-50° C. and stirred for 20 min at the same temperature. Filtered the reaction mixture and washed with hot methanol solution. Distilled off the solvent completely from the filtrate under reduced pressure to get 4-amino-3,4-dihydroquinoxalin-2(1H)-one compound of formula-6.

Step-b: Methanol (1600 ml) was added to compound of formula-6 obtained in step-a) at 25-30° C. Ethyl 4-oxopiperidine-1-carboxylate compound of formula-7a (154.6 gm) was added to the reaction mixture at 25-30° C. Acidified the reaction mixture by adding methanolic HCl solution at 25-30° C. and stirred for 1 hr 35 min at the same temperature. Filtered the solid, washed with methanol and dried to get the title compound. Yield: 196 gm.

### Example-4: Preparation of ethyl 2-oxo-2,3,6b,7,10,10a-hexahydro-1H-pyrido[3′,4′:4,5]pyrrolo[1,2,3-de]quinoxaline-8(9H)-carboxylate (Formula-9a)

A mixture of trifluoroacetic acid (600 ml) and dichloromethane (1400 ml) was cooled to −10° C. to −5° C. and stirred for 5 min at the same temperature. Compound of formula-8a (200 gm) was added to the reaction mixture at −10° C. to −5° C. and stirred for 10 min at the same temperature. Sodium borohydride (20.24 gm) was slowly added lot wise to the reaction mixture for 45 min at −10° C. to −5° C. and stirred for 2 hr 10 min at the same temperature. 20% Aqueous sodium carbonate solution was slowly added drop wise to the reaction mixture at −10° C. to −5° C. and stirred for 15 min at the same temperature. Water and dichloromethane were added to the reaction mixture at −10° C. to −5° C. and stirred for 10 min at the same temperature. Raised the temperature of the reaction mixture to 25-30° C. Both the organic and aqueous layers were separated and extracted the aqueous layer with dichloromethane. Combined the organic layers and washed with 2% aqueous sodium carbonate solution. Distilled off the solvent completely from the organic layer and co-distilled with isopropyl alcohol. Isopropyl alcohol (1000 ml) was added to the obtained compound at 25-30° C. Heated the mixture to 55-60° C. and stirred for 20 min at the same temperature. Cooled the mixture to 25-30° C. and stirred for 3 hr at the same temperature. Filtered the solid, washed with isopropyl alcohol and dried to get the title compound. Yield: 169 gm.

### Example-5: Preparation of ethyl 3-methyl-2-oxo-2,3,6b,7,10,10a-hexahydro-1H-pyrido[3′,4′:4,5]pyrrolo[1,2,3-de]quinoxaline-8(9H)-carboxylate (Formula-10a)

Potassium tert.butoxide (37.1 gm) followed by methyl iodide (20.4 ml) were added to a mixture of dimethylformamide (700 ml) and compound of formula-9a (100 gm) at 20-30° C. and stirred the reaction mixture for 1 hr at the same temperature. Potassium tert.butoxide (26 gm) and methyl iodide (14.3 ml) were added to the reaction mixture at 20-25° C. and stirred for 1 hr 20 min at the same temperature. Water (3500 ml) was added to the reaction mixture at 25-30° C. and stirred for 30 min at the same temperature. Filtered the solid, washed with water. Dichloromethane followed by water were added to the solid at 25-30° C. Both the organic and aqueous layers were separated and dried the organic layer over sodium sulfate. Distilled off the solvent completely from the organic layer and co-distilled with methyl tert.butyl ether. Methyl tert.butyl ether (500 ml) was added to the obtained compound at 25-30° C. Heated the mixture to 50-55° C. and stirred for 25 min at the same temperature. Cooled the mixture to 25-30° C. and stirred for 90 min at the same temperature. Filtered the solid, washed with methyl tert.butyl ether and dried to get title compound. Yield: 78 gm.

### Example-6: Preparation of 3-methyl-6b,7,8,9,10,10a-hexahydro-1H-pyrido[3′,4′:4,5]pyrrolo[1,2,3-de]quinoxalin-2(3H)-One (Formula-11)

A mixture of compound of formula-10a (30 gm), potassium hydroxide (63.9 gm) and n-butanol (330 ml) was heated to 105-110° C. and stirred for 5 hr 45 min at the same temperature. Distilled off the solvent from the reaction mixture under reduced pressure. Cooled the obtained compound to 25-30° C., water and ethyl acetate were added to it and stirred the mixture for 10 min at the same temperature. Both the organic and aqueous layers were separated and extracted the aqueous layer with ethyl acetate. Combined the organic layers and washed with water. Distilled off the solvent completely from the organic layer to get the title compound. Yield: 15 gm.

### Example-7a: Preparation of (6bR,10aS)-3-methyl-6b,7,8,9,10,10a-hexahydro-1H-pyrido [3′,4′:4,5]pyrrolo[1,2,3-de]quinoxalin-2(3H)-One (Formula-14)

Step-a: A mixture of compound of formula-11 (1.5 gm) and ethanol (15 ml) was stirred for 5 min at 25-30° C. (2S,3S)-2,3-bis(benzoyloxy)-4-(dimethylamino)-4-oxobutanoic acid compound of formula-12a (1.66 gm) was added to the reaction mixture at 25-30° C. and stirred for 8 hr at the same temperature. Filtered the solid, washed with ethanol and dried. Ethanol (10 ml) was added to the obtained compound at 25-30° C. Heated the mixture to 75-80° C. and stirred for 30 min at the same temperature. Ethanol (15 ml) was slowly added to the mixture at 75-80° C. and stirred for 3 hr at the same temperature. Cooled the mixture to 25-30° C. and stirred for 8 hr at the same temperature. Filtered the solid, washed with ethanol and dried to get (6bR,10aS)-3-methyl-6b,7,8,9,10,10a-hexahydro-1H-pyrido[3′,4′:4,5]pyrrolo [1,2,3-de]quinoxalin-2(3H)-one (2S,3S)-2,3-bis(benzoyloxy)-4-(dimethylamino)-4-oxobutanoate compound of formula-13a.
Step-b: Water (15 ml) was added to compound of formula-13a obtained in step-a) at 25-30° C. 2N Aqueous sodium hydroxide solution (10 ml) was slowly added drop wise to the reaction mixture at 25-30° C. and stirred the reaction mixture for 5 min at the same temperature. Ethyl acetate and tetrahydrofuran were added to the reaction mixture at 25-30° C. and stirred for 5 min at the same temperature. Both the organic and aqueous layers were separated and extracted the aqueous layer with a mixture of ethyl acetate and tetrahydrofuran. Combined the organic layers, washed with water and dried over sodium sulfate. Distilled off the solvent completely from the organic layer to get the title compound. Yield: 400 mg.

### Example-7b: Preparation of Compound of Formula-14

Step-a: Compound of formula-12a (1.66 gm) was added to a mixture of compound of formula-11 (1.5 gm) and acetonitrile (15 ml) at 25-30° C. and stirred the reaction mixture for 40 min at the same temperature. Acetonitrile (15 ml) was added to the reaction mixture at 25-30° C. and stirred for 7 hr 10 min at the same temperature. Filtered the solid, washed with acetonitrile and dried. Acetonitrile (10 ml) was added to the obtained compound at 25-30° C. Heated the mixture to 75-85° C. and stirred for 25 min at the same temperature. Acetonitrile (110 ml) was added lot wise to the mixture at 75-85° C. and stirred for 4 hr 20 min at the same temperature. Cooled the mixture to 25-30° C. and stirred for 8 hr at the same temperature. Filtered the solid, washed with acetonitrile and dried to get compound of formula-13a.

Step-b: Water (15 ml) was added to compound of formula-13a at 25-30° C. 2N Aq. sodium hydroxide solution (10 ml) was slowly added drop wise to the reaction mixture at 25-30° C. Ethyl acetate and tetrahydrofuran were added to the reaction mixture at 25-30° C. and stirred the reaction mixture for 5 min at the same temperature. Both the organic and aqueous layers were separated and extracted the aqueous layer with a mixture of ethyl acetate and tetrahydrofuran. Washed the organic layer with water and dried over sodium sulfate. Distilled off the solvent completely from the organic layer to get the title compound. Yield: 400 mg.

### Example-8: Preparation of (6bR,10aS)-3-methyl-2,3,6b,7,8,9,10,10a-octahydro-1H-pyrido[3′,4′:4,5]pyrrolo[1,2,3-de]quinoxaline (Formula-15)

A mixture of compound of formula-14 (4 gm) and tetrahydrofuran (20 ml) was stirred for 10 min at 25-30° C. Borane-dimethylsulfide (6 ml) was slowly added to the reaction mixture at 25-30° C. and stirred for 10 min at the same temperature. Heated the reaction mixture to 60-65° C. and stirred for 2 hr 20 min at the same temperature. Cooled the reaction mixture to 5-10° C. Methanol (6 ml) was slowly added to the reaction mixture at 5-10° C. and stirred for 20 min at the same temperature. 6N Aqueous hydrochloric acid solution (6 ml) was slowly added to the reaction mixture at 5-10° C. and stirred for 5 min at the same temperature. Heated the reaction mixture to 40-45° C. and stirred for 45 min at the same temperature. Water was added to the reaction mixture at 40-45° C. Cooled the reaction mixture to 25-30° C., basified it with 20% aqueous sodium carbonate solution and stirred for 20 min at the same temperature. Ethyl acetate was added to the reaction mixture at 25-30° C. and stirred for 10 min at the same temperature. Both the organic land aqueous layers were separated and extracted the aqueous layer with ethyl acetate. Combined the organic layers and dried over sodium sulfate. Filtered the organic layer and distilled off the solvent completely from the organic layer. Ethyl acetate (40 ml) and isopropyl alcohol-HCl (8 ml) were added to the obtained compound at 25-30° C. and stirred for 20 min at the same temperature. Filtered the solid and washed with ethyl acetate. Water (40 ml) and 2N aqueous sodium hydroxide solution (5 ml) were added to the obtained compound at 25-30° C. and stirred the mixture for 15 min at the same temperature. Extracted the compound into ethyl acetate. Separated the organic and aqueous layers and washed the organic layer with water. Distilled off the solvent from the organic layer to get the title compound. Yield: 2.3 gm.

### Example-9: Preparation of Compound of Formula-1

Triethylamine (4.4 gm), potassium iodide (2.89 gm) followed by 4-chloro-1-(4-fluorophenyl)butan-1-one compound of formula-16a (2.09 gm) were added to a solution of compound of formula-15 (2 gm), toluene (11 ml) and 1,4-dioxane (11 ml) at 25-30° C. and stirred the reaction mixture for 20 min at the same temperature. Heated the reaction mixture to 100-105° C. and stirred for 5 hr 45 min at the same temperature. Cooled the reaction mixture to 25-30° C. and stirred for 11 hr at the same temperature. Distilled off the solvent completely from the reaction mixture. Cooled the obtained compound to 25-30° C., water and ethyl acetate were added to it and stirred for 5 min at the same temperature. Both the organic and aqueous layers were separated and extracted the aqueous layer with ethyl acetate. Combined the organic layers and water (10 ml) was added to it at 25-30° C. Cooled the reaction mixture to 5-10° C. 6N Aqueous hydrochloric acid solution was slowly added drop wise to the reaction mixture at 5-10° C. and stirred for 5 min at the same temperature. Ethyl acetate was added to the reaction mixture at 5-10° C. and raised the temperature of the reaction mixture to 25-30° C. Both the organic and aqueous layers were separated and washed the aqueous layer with ethyl acetate. Basified the aqueous layer with 2N aqueous sodium hydroxide solution at 25-30° C. and stirred the reaction mixture for 15 min at the same temperature. Ethyl acetate was added to the reaction mixture at 25-30° C. and stirred for 5 min at the same temperature. Both the organic and aqueous layers were separated and extracted the aqueous layer with ethyl acetate. Combined the organic layers, charcoal (0.2 gm) was added to it at 25-30° C. and stirred for 25 min at the same temperature. Filtered the reaction mixture through hyflow bed and washed the hyflow bed with ethyl acetate. Dried the organic layer over sodium sulfate and distilled off the solvent to get title compound. Yield: 1.7 gm.

### Example-10: Preparation of Compound of Formula-1a

Compound of formula-1 (1.5 gm) was dissolved in isopropyl alcohol (11 ml) at 25-30° C. and stirred the solution for 5 min at the same temperature. A solution of p-toluenesulfonic acid (0.65 gm) in isopropyl alcohol (5 ml) was added to the reaction mixture at 25-30° C. and stirred for 1 hr 10 min at the same temperature. Compound of formula-1a was added as seed material to the reaction mixture at 25-30° C. and stirred for 16 hr 10 min at the same temperature. Filtered the solid, washed with isopropyl alcohol and dried to get the title compound. Yield: 300 mg.

### Example-11: Preparation of (6bR,10aS)-3-methyl-6b,7,8,9,10,10a-hexahydro-1H-pyrido[3′,4′:4,5]pyrrolo[1,2,3-de]quinoxalin-2(3H)-one (2S,3S)-2,3-dihydroxysuccinate

Methanol (100 ml) was added to compound of formula-11 (10 gm) at 25-30° C. and stirred the mixture for 10 min at the same temperature. Heated the mixture to 60-65° C., D-(−)-tartaric acid (2.46 gm) was added to it and stirred the reaction mixture at the same temperature. Cooled the reaction mixture to 35-40° C. Filtered the reaction mixture, washed with methanol and dried. Methanol (100 ml) was added to the obtained compound at 25-30° C. and stirred at the same temperature. Heated the mixture to 60-65° C. and stirred for 50 min at the same temperature. Cooled the mixture to 25-30° C. Filtered the solid, washed with methanol and dried to get the title compound. Yield: 3.2 gm.

### Example-12: Preparation of (6bR,10aS)-3-methyl-2,3,6b,7,8,9,10,10a-octahydro-1H-pyrido[3′,4′:4,5]pyrrolo[1,2,3-de]quinoxaline hydrochloride (Formula-15a)

Dichloromethane (300 ml) and water (500 ml) were added to (6bR,10aS)-3-methyl-6b,7,8,9,10,10a-hexahydro-1H-pyrido[3′,4′:4,5]pyrrolo[1,2,3-de]quinoxalin-2(3H)-one (2S,3S)-2,3-dihydroxysuccinate at 25-30° C. and stirred at the same temperature. Adjusted the reaction mixture pH to 12-13 with aqueous sodium hydroxide solution at 25-30° C. and stirred at the same temperature. Separated both organic and aqueous layers. Dichloromethane (200 ml) was added to the aqueous layer at 25-30° C. and stirred at the same temperature. Separated both organic and aqueous layers. Combined both the organic layers and distilled off the solvent completely from organic layer under reduced pressure. Tetrahydrofuran (500 ml) was added to the obtained compound at 25-30° C. and stirred at the same temperature. Borane dimethylsulfide (50.8 ml) was added to the reaction mixture at 25-30° C. and stirred at the same temperature. Heated the reaction mixture to 60-65° C. and stirred at the same temperature. Cooled the reaction mixture to 25-30° C. and methanol (150 ml) was added drop wise at the same temperature. Aqueous hydrochloric acid solution (75 ml of HCl in 75 ml water) was added to the reaction mixture at 25-30° C. Heated the reaction mixture to 40-45° C. and stirred for 45 min at the same temperature. Cooled the reaction mixture to 25-30° C. Toluene and water were added to the reaction mixture at 25-30° C. and stirred at the same temperature. Adjusted the reaction mixture pH to 8 with aqueous sodium carbonate solution at 25-30° C. and stirred at the same temperature. Separated both organic and aqueous layers. Aqueous layer extracted with toluene. Combined the organic layers. Distilled off the solvent completely from organic layer under reduced pressure. Isopropanol (300 ml) was added to the obtained compound at 25-30° C. and stirred at the same temperature. Isopropanolic hydrochloric acid (80 ml) was added to the above mixture at 25-30° C. and stirred at the same temperature. Filtered the solid, washed with isopropanol and dried to get the title compound. Yield: 62 gm.

### Example-13: Preparation of Compound of Formula-4

A mixture of water (500 ml) and sodium bicarbonate (116.5 gm) was stirred for 5 min at 25-30° C. 2-Chloroacetic acid (104.9 gm) was slowly added to the reaction mixture at 25-30° C. and stirred for 20 min at the same temperature. Benzene-1,2-diamine (100 gm) was added to the reaction mixture at 25-30° C. Heated the reaction mixture to 90-95° C. and stirred for 4 hr 30 min at the same temperature. Cooled the reaction mixture to 0-5° C. and stirred for 3 hr at the same temperature. Filtered the solid, washed with water and dried to get the title compound. Yield: 89 gm.

### Example-14: Preparation of Compound of Formula-5

Compound of formula-4 (100 gm) was added to a mixture of acetic acid (600 ml) and water (100 ml) at 25-30° C. Cooled the reaction mixture to 0-5° C. Slowly added sodium nitrite solution (60.5 gm sodium nitrite dissolved in 100 ml water) to the reaction mixture at 0-5° C. and stirred for 5 hr at the same temperature. Filtered the solid, washed with water and dried to get the title compound. Yield: 96 gm.

### Example-15: Preparation of Compound of Formula-8a

A mixture of methanol (2000 ml) and ammonium chloride (120.5 gm) was stirred for 5 min at 25-30° C. Compound of formula-5 (100 gm) was added to a reaction mixture at 25-30° C. and stirred for 5 min at the same temperature. Cooled the reaction mixture to 15-20° C. Zinc dust (147.94 gm) was slowly added lot wise to the reaction mixture at 15-20° C. over a period of 1 hr 30 min under stirring at the same temperature. Raised the temperature of the reaction mixture to 25-30° C. and stirred for 45 min at the same temperature. Ammonium chloride (30 gm) was added to the reaction mixture at 25-30° C. and stirred for 3 hr at the same temperature. Heated the reaction mixture to 45-50° C. and stirred for 30 min at the same temperature. Filtered the reaction mixture and washed with methanol. Distilled off the solvent completely from the filtrate. Methanol (800 ml) and compound of formula-7a (77.3 gm) were added to the obtained compound at 25-30° C. and stirred for 20 min at the same temperature. Methanolic HCl (150 ml) was added to the reaction mixture at 25-30° C. and stirred for 2 hr at the same temperature. Filtered the solid, washed with methanol and dried to get the title compound. Yield: 102 gm.

### Example-16: Preparation of Compound of Formula-10a

Trifluoroacetic acid (186 gm) was added to a pre-cooled mixture of compound of formula-8a (50 gm) and dichloromethane (350 ml) at −5° C. to −10° C. under nitrogen atmosphere and stirred for 15 min at the same temperature. Sodium borohydride (4.98 gm) was slowly added lot wise to the reaction mixture at −5° C. to −10° C. and stirred for 2 hr 30 min at the same temperature. Quenched the reaction mixture with aqueous ammonia solution at 0-5° C. Raised the temperature of the reaction mixture to 25-30° C. and filtered the reaction mixture through hyflow bed. Dichloromethane was added to the filtrate at 25-30° C. and stirred for 10 min at the same temperature. Both the organic and aqueous layers were separated and extracted the aqueous layer with dichloromethane. Combined the organic layers and washed with aqueous sodium chloride solution followed by with water. Distilled off the solvent from the organic layer and co-distilled with isopropyl alcohol. Isopropyl alcohol (300 ml) was added to the obtained compound at 25-30° C. and stirred the mixture for 5 min at the same temperature. Heated the mixture to 50-55° C. and stirred for 30 min at the same temperature. Cooled the mixture to 25-30° C. and stirred for 3 hr at the same temperature. Filtered the solid, washed with isopropyl alcohol and dried. Yield: 37 gm.

Acetone (500 ml) was added to compound obtained (35 gm) at 25-30° C. Potassium tert.butoxide (19.5 gm) was added lot wise to the reaction mixture at 25-30° C. and stirred for 20 min at the same temperature. Dimethyl sulfate (16 ml) was slowly added to the reaction mixture at 25-30° C. and stirred for 2 hr 30 min at the same temperature. Water (1000 ml) was slowly added to the reaction mixture at 25-30° C. and stirred for 2 hr at the same temperature. Filtered the solid, washed with water and dried to get title compound. Yield: 33 gm.

### Example-17: Preparation of Compound of Formula-11

A mixture of n-butanol (800 ml) and potassium hydroxide (142 gm) was stirred for 20 min at 25-30° C. Compound of formula-10a (100 gm) was added to the reaction mixture at 25-30° C. and stirred for 10 min at the same temperature. Heated the reaction mixture to 100-105° C. and stirred for 4 hr at the same temperature. Cooled the reaction mixture to 25-30° C. Filtered the reaction mixture through hyflow bed and washed with n-butanol. Cooled the filtrate to 10-15° C., acidified it with 20% isopropyl alcohol-HCl (400 ml) and stirred for 2 hr at the same temperature. Filtered the solid and washed with n-butanol. Water (500 ml) was added to the obtained compound at 25-30° C. and basified the reaction mixture by using aqueous sodium carbonate solution. Dichloromethane (800 ml) and charcoal (10 gm) were added to the reaction mixture at 25-30° C. and stirred for 25 min at the same temperature. Filtered the reaction mixture through hyflow bed and washed with dichloromethane. Both the organic and aqueous layers were separated and extracted the aqueous layer with dichloromethane. Combined the organic layers and washed with water. Distilled off the solvent completely from the organic layer and co-distilled with methyl tert.butyl ether. Methyl tert.butyl ether (300 ml) was added to the obtained compound at 25-30° C. Heated the mixture to 50-55° C. and stirred for 50 min at the same temperature. Cooled the mixture to 0-5° C. and stirred for 4 hr at the same temperature. Filtered the solid, washed with methyl tert.butyl ether and dried to get the title compound. Yield: 53.2 gm.

### Example-18: Preparation of (6bR,10aS)-3-methyl-6b,7,8,9,10,10a-hexahydro-1H-pyrido[3′,4′:4,5]pyrrolo[1,2,3-de]quinoxalin-2(3H)-one (2S,3S)-2,3-dihydroxysuccinate

A mixture of compound of formula-11 (10 gm) and methanol (100 ml) was stirred for 10 min at 25-30° C. Heated the reaction mixture to 60-65° C. D(−)-Tartaric acid (4.93 gm) was added to the reaction mixture at 60-65° C. and stirred for 1 hr at the same temperature. Gradually cooled the reaction mixture to 35-40° C. and stirred for 5 min at the same temperature. Filtered the solid and washed with methanol. Methanol (80 ml) was added to the obtained compound at 25-30° C. and stirred for 15 min at the same temperature. Heated the mixture to 60-65° C. and stirred for 1 hr at the same temperature. Cooled the mixture to 35-40° C., filtered the solid and washed with methanol. Repeated the purification process twice with methanol and dried the obtained compound to get title compound. Yield: 3.8 gm.

### Example-19: Preparation of (6bR,10aS)-3-methyl-2,3,6b,7,8,9,10,10a-octahydro-1H-pyrido[3′,4′:4,5]pyrrolo[1,2,3-de]quinoxaline dihydrobromide (Formula-15b)

Step-a: A mixture of (6bR,10aS)-3-methyl-6b,7,8,9,10,10a-hexahydro-1H-pyrido[3′,4′:4,5]pyrrolo[1,2,3-de]quinoxalin-2(3H)-one (2S,3S)-2,3-dihydroxysuccinate (25 gm), dichloromethane (125 ml) and water (125 ml) was stirred for 10 min at 25-30° C. Basified the reaction mixture with 20% aqueous sodium carbonate solution at 25-30° C. and stirred for 15 min at the same temperature. Both the organic and aqueous layers were separated and extracted the aqueous layer with dichloromethane. Combined the organic layers and washed with water. Distilled off the solvent completely from the organic layer. 1,2-Dimethoxyethane (50 ml) was added to the obtained compound at 40-45° C. Heated the mixture to 75-80° C. and stirred for 20 min at the same temperature. Distilled off the solvent completely from the mixture. Cyclohexane (50 ml) was added to the obtained compound at 25-30° C. and stirred for 35 min at the same temperature. Filtered the solid, washed with cyclohexane and dried.
Step-b: Aluminium chloride (10 gm) was added lot wise to 1,2-dimethoxyethane (75 ml) at 5-10° C. and stirred the mixture for 1 hr at the same temperature. Sodium borohydride (7.2 gm) was added lot wise to the reaction mixture at 5-10° C. and stirred for 1 hr at the same temperature. The compound obtained in step-a) (15.5 gm) was slowly added lot wise to the reaction mixture at 5-10° C. and stirred for 35 min at the same temperature. Raised the temperature of the reaction mixture to 25-30° C. and stirred for 9 hr 30 min at the same temperature. Quenched the reaction mixture with pre-cooled water at 0-5° C. and stirred for 30 min at the same temperature. Raised the temperature of the reaction mixture to 25-30° C. and stirred for 1 hr at the same temperature. Distilled off the solvent from the reaction mixture. Aqueous hydrochloric acid solution was added to the obtained compound at 25-30° C. and stirred for 1 hr at the same temperature. Heated the reaction mixture to 70-75° C. and stirred for 1 hr at the same temperature. Cooled the reaction mixture to 25-30° C. and basified with 20% aqueous sodium hydroxide solution. Toluene was added to the reaction mixture at 25-30° C. and stirred for 15 min at the same temperature. Both the organic and aqueous layers were separated and the aqueous layer was extracted with toluene. Combined the organic layers and washed with water. Distilled off the solvent completely from the organic layer under reduced temperature. Isopropyl alcohol (125 ml) was added to the obtained compound at 25-30° C. and stirred the mixture for 30 min at the same temperature. HBr in acetic acid (20 ml) was added to the mixture at 25-30° C. and stirred for 1 hr at the same temperature. Filtered the solid, washed with isopropyl alcohol and dried to get the title compound. Yield: 18 gm.

### Example-20: Preparation of Compound of Formula-1a

Step 1: Preparation of 4-bromo-1-(4-fluorophenyl)butan-1-one (Formula-16b)

A mixture of sodium hydroxide (9 gm) and water (150 ml) was stirred for 15 min at 25-30° C. 4-Chloro-1-(4-fluorophenyl)butan-1-one compound of formula-16a (30 gm) and tetrahydrofuran (60 ml) were added to the mixture at 25-30° C. Heated the reaction mixture to 60-65° C. and stirred for 3 hr 15 min at the same temperature. Water and toluene were added to the reaction mixture at 25-30° C. and stirred for 10 min at the same temperature. Both the organic and aqueous layers were separated and extracted the aqueous layer with toluene. Combined the organic layers and washed with water. Distilled off the solvent completely from the organic layer under reduced pressure. 40% Aqueous hydrobromic acid (75 ml) was added to the obtained compound at 25-30° C. and stirred for 10 min at the same temperature. Heated the reaction mixture to 80-85° C. and stirred for 5 hr at the same temperature. Water and toluene were added to the reaction mixture at 25-30° C. and stirred for 15 min at the same temperature. Both the organic and aqueous layers were separated and extracted the aqueous layer with toluene. Combined the organic layers and washed with sodium thiosulfate and water. Distilled off the solvent completely from the organic layer under reduced pressure to get the title compound. Yield: 29 gm.

### Step 2: Preparation of Compound of Formula-1a

A mixture of compound of formula-15b (50 gm) and acetone (400 ml) was stirred for 10 min at 25-30° C. Potassium carbonate (61.7 gm), potassium iodide (38.2 gm) and compound of formula-16b (47 gm) were added to the reaction mixture at 25-30° C. and stirred for 4 hr at the same temperature. Water and toluene were added to the reaction mixture at 25-30° C. and stirred for 15 min at the same temperature. Both the organic and aqueous layers were separated and the aqueous layer was extracted with toluene. Combined the organic layers and washed with water. Water was added to the organic layer at 25-30° C. and stirred for 20 min at the same temperature. Aqueous HCl solution (25 ml HCl dissolved in 125 ml water) was added to the reaction mixture at 25-30° C. and stirred for 20 min at the same temperature. Both the organic and aqueous layers were separated. Toluene was added to the aqueous layer at 25-30° C. Cooled the reaction mixture to 15-20° C., basified the reaction mixture with aqueous sodium hydroxide solution and stirred for 20 min at the same temperature. Raised the temperature of the reaction mixture to 25-30° C. and stirred for 15 min at the same temperature. Both the organic and aqueous layers were separated and extracted the aqueous layer with toluene. Combined the organic layers and washed with water. Charcoal (5 gm) was added to the organic layer at 25-30° C. and stirred for 25 min at the same temperature. Filtered the reaction mixture through hyflow bed and washed with toluene. Distilled off the solvent completely from the filtrate under reduced pressure. Isopropyl alcohol (250 ml) and p-toluenesulfonic acid solution (19.4 gm p-toluenesulfonic acid dissolved in 150 ml isopropanol) were added to the obtained compound at 25-30° C. and stirred for 7 hr at the same temperature. Filtered the solid, washed with isopropyl alcohol and dried to get the title compound. Yield: 42 gm.

### Example-21: Purification of Compound of Formula-1a

A mixture of compound of formula-1a (40 gm) and dichloromethane (320 ml) was stirred for 15 min at 25-30° C. Charcoal (2 gm) was added to the mixture at 25-30° C. and stirred for 40 min at the same temperature. Filtered the mixture through hyflow bed and washed with dichloromethane. Distilled off the solvent completely from the filtrate under reduced pressure and co-distilled with isopropyl alcohol. Isopropyl alcohol (280 ml) was added to the obtained compound at 25-30° C. and stirred for 10 min at the same temperature. Heated the mixture to 80-85° C. and stirred for 10 min at the same temperature. Cooled the mixture to 25-30° C. and stirred for 2 hr at the same temperature. Filtered the solid, washed with isopropyl alcohol and dried to get the title compound.

The PXRD pattern of the obtained compound is similar to the PXRD pattern of crystalline Form-A of compound of formula-1a described in U.S. Pat. No. 8,648,077B2.

### Yield: 35.2 gm.

We claim:

1. A process for the preparation of compound of formula-14 comprising resolution of compound of formula-11.

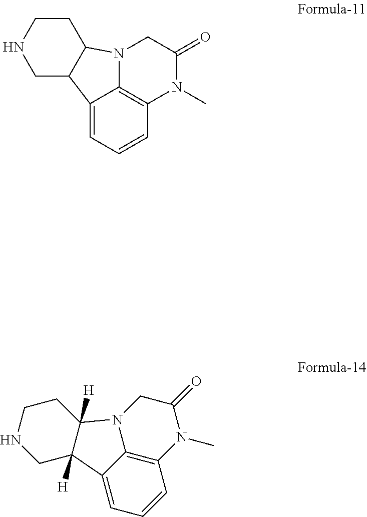 [SMILES: CN1C(=O)CN2c3c(cccc31)C1CNCCC12 ; CN1C(=O)CN2c3c(cccc31)[C@@H]1CNCC[C@@H]12]

2. The process according to claim 1, wherein the resolution is carried out using a chiral acid in a solvent to provide corresponding diastereomeric salt.

3. The process according to claim 2, further comprising treating the diastereomeric salt with a base in a solvent to provide compound of formula-14.

4. The process according to claim 2, wherein;
the chiral acid is selected from (2S,3S)-2,3-bis(benzoyloxy)-4-(dimethylamino)-4-oxobutanoic acid, (2S,3S)-2,3-bis(benzoyloxy)-4-(diethylamino)-4-oxobutanoic acid, (2S,3S)-2,3-bis(benzoyloxy)-4-oxo-4-(pyrrolidin-1-yl)butanoic acid, (2S,3S)-4-(isopropylamino)-2,3-bis((4-methylbenzoyl)oxy)-4-oxo butanoic acid, (2S,3S)-2,3-bis (benzoyloxy)-4-(isopropylamino)-4-oxobutanoic acid, mandelic acid, acetyl mandelic acid, tartaric acid, di-p-tolyl tartaric acid, dibenzoyl tartaric acid, camphor sulfonic acid;
the solvent is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

5. The process according to claim 3, wherein;
the base is selected from inorganic bases and organic bases; and
the solvent is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixture of any of the afore mentioned solvents.

6. The process according to claim 2, wherein the chiral acid has the structural formula 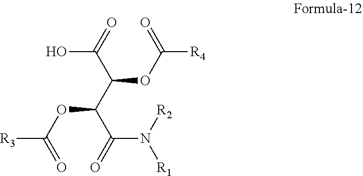
wherein, ‘R₁’ and ‘R₂′ are independently H, lower alkyl, or lower cycloalkyl; or ‘R₁’ and ‘R₂’ together with the Nitrogen atom to which they are attached form an optionally substituted 3-6 membered saturated heterocyclic ring optionally containing 1 or 2 additional heteroatoms selected from S or O; ‘R₃’ and ‘R₄’ are independently phenyl or 5-6 membered heteroaryl and is optionally substituted by halogen, hydroxyl, nitro, lower alkyl, or lower cycloalkyl.

7. The process according to claim 1 further comprising;
a) reducing compound of formula-14 with a reducing agent in a solvent to provide compound of formula-15, which is optionally converted to its acid-addition salt by treating with an acid in a solvent, 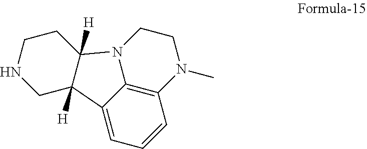 [SMILES: CN1CCN2c3c(cccc31)[C@@H]1CNCC[C@@H]12]
b) reacting compound of formula-15 or its salt with compound of formula-16 to provide compound of formula-1, 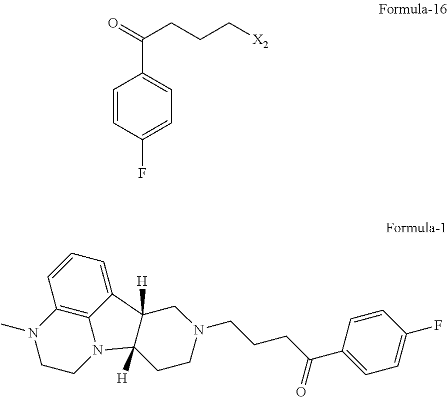
wherein, ‘X₂’ selected from halogen such as F, Cl, Br, I;
c) treating compound of formula-1 with an acid in a solvent to provide corresponding acid-addition salt.

8. The process according to claim 7, wherein;
the reducing agent in step-a) is selected from Borane-Dimethyl sulfide (Borane-DMS), Borane-Tetrahydrofuran (Borane-THF), LiAlH₄, NaBH₄, sodium cyanoborohydride, Aluminium hydride (AlH₃), lithium trialkoxyaluminium hydrides such as lithium triethoxyaluminium hydride (Li(EtO)3AlH), lithium tri tert.butoxyaluminium hydride (Li(OtBu)3AlH), diisobutylaluminium hydride (DIBAL), tetramethylammonium triacetoxyborohydride, triethylsilane, NaAlH(O-t-Bu)₃, Na(AcO)₃BH, B₂H₆, sodium bis(2-methoxyethoxy)aluminumhydride (Red-Al or Vitride), catalytic hydrogenation in presence of Pd, Pt, Rh, Zn, Raney Ni, PtO₂ and the like; Fe, Fe in acidic media like NH₄Cl, HCl, acetic acid; Sn in acidic media like HCl;
Zn, Zn in acidic media like HCl, NH₄Cl, acetic acid, formic acid or ammonium formate;
the acid is selected from inorganic acids.

9. The process according to claim 7 wherein step-b) is carried out in presence of a base selected from organic bases and inorganic bases or mixtures thereof optionally in presence of alkali metal halide.

10. The process according to claim 7, wherein the acid in step-c) is selected from inorganic and organic acids; preferably p-toluenesulfonic acid.

11. The process according to claim 7, wherein the solvent in step-a) to step-c) wherever necessary is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixtures thereof.

12. Compounds having the structural formulae;

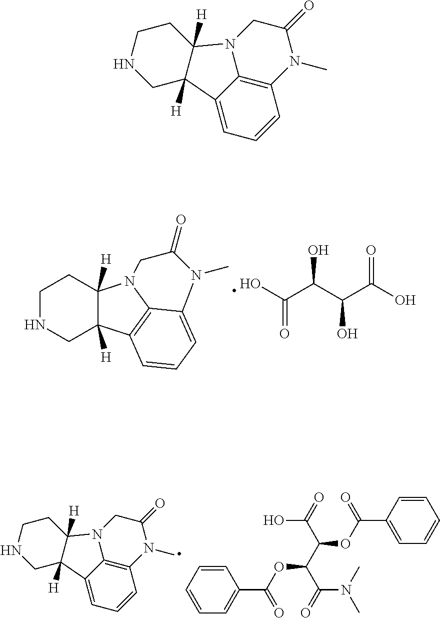

13. Use of compounds according to claim 12 for the preparation of Lumateperone or its pharmaceutically acceptable salts.

14. A process for the preparation of compound of formula-15, 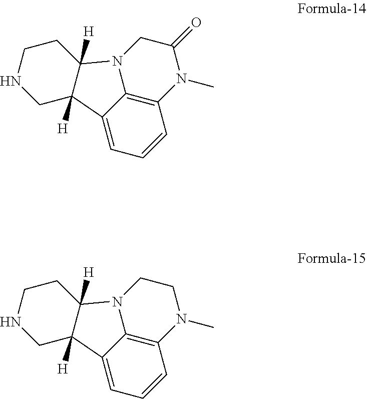 [SMILES: CN1C(=O)CN2c3c(cccc31)[C@@H]1CNCC[C@@H]12 ; CN1CCN2c3c(cccc31)[C@@H]1CNCC[C@@H]12]
comprising reducing compound of formula-14 with a reducing agent in a solvent.

15. The process according to claim 14, wherein the reducing agent is selected from Borane-Dimethyl sulfide (Borane-DMS), Borane-Tetrahydrofuran (Borane-THF), Lithium aluminium hydride (LiAlH₄), NaBH₄, sodium cyanoborohydride, Aluminium hydride (AlH₃), lithium trialkoxyaluminium hydrides such as lithium triethoxyaluminium hydride (Li(EtO)3AlH), lithium tri tert.butoxyaluminium hydride (Li(OtBu)3AlH), diisobutylaluminium hydride (DIBAL), tetramethylammonium triacetoxyborohydride, triethylsilane, NaAlH(O-t-Bu)₃, Na(AcO)₃BH, B₂H₆, sodium bis(2-methoxyethoxy)aluminumhydride (Red-Al or Vitride), catalytic hydrogenation in presence of Pd, Pt, Rh, Zn, Raney Ni, PtO₂ and the like; Fe, Fe in acidic media like NH₄Cl, HCl, acetic acid; Sn in acidic media like HCl; Zn, Zn in acidic media like HCl, NH₄Cl, acetic acid, formic acid or ammonium formate.

16. The process according to claim 14, wherein the solvent is selected from hydrocarbon solvents, ether solvents, ester solvents, polar-aprotic solvents, chloro solvents, ketone solvents, nitrile solvents, alcohol solvents, polar solvents, formic acid, acetic acid and the like or mixtures thereof.
